(19) United States
(12) Patent Application Publication
(10) Pub. No.: US 2013/0327324 A1
(43) Pub. Date: Dec. 12, 2013

(54) DEVICE COMPRISING A SET OF MAGNETIC ELEMENTS PARTICULARLY SUITABLE AS A SUPPORT TO THE PROCEDURE OF PATIENT'S TRACHEAL INTUBATION

(71) Applicant: Andrea De Domenico, Rome (IT)

(72) Inventor: Andrea De Domenico, Rome (IT)

(21) Appl. No.: 14/001,097

(22) PCT Filed: Feb. 1, 2012
(86) PCT No.: PCT/EP12/51715

§ 371 Date: Aug. 22, 2013

(30) Foreign Application Priority Data
Feb. 23, 2011 (EP) 11425045.9

(51) Int. Cl. A61M 16/04; A61B 1/267

(52) U.S. Cl. CPC A61M 16/0488; A61B 1/267
USPC 128/200.26

(57) ABSTRACT

A device particularly suitable as a support to the procedure of tracheal intubation, preferably embedded in a blade of a laryngoscope or similar instruments, and having two sets of flexible magnetic elements. The first set of magnetic elements being free to move forward or backward, under control of an operator, along the longitudinal direction of an operative channel belonging to the same device. The second set of magnetic elements is inserted in an endotracheal tube. The sets are aligned such that they have opposite polarity, whereby they can be used to guide and route the endotracheal tube.

### CROSS REFERENCE TO RELATED APPLICATIONS

This patent application is a national phase entry under 35 U.S.C. §371 of PCT International Patent Application PCT/EP2012/051715, filed Feb. 1, 2012, designating the United States and published in English as International Patent Publication WO 2012/113628 A1 on Aug. 30, 2012, which claims the benefit, under Article 8 of the PCT, of European Patent Application Serial No. 11425045.9, filed Feb. 23, 2011, the benefit and entire disclosure of each of which is hereby incorporated herein by this reference.

### FIELD OF THE INVENTION

The present invention concerns a medical device, that is particularly suitable as a support to the procedure of patient's tracheal intubation, and preferably embedded in a blade of a laryngoscope, or an Airtraq® video-laryngoscope, or a Glidescope® video-laryngoscope, or a LMA® video-laryngoscope, or other similar instruments, and that comprises magnetic means able to achieve a guiding and routing system of an endotracheal tube, towards its final position with a far end in trachea.

### BACKGROUND OF THE INVENTION

Direct laryngoscopy is a technique that permits one to visualize the laryngeal structures in a clear and straight way, and it is achieved by an instrument called a laryngoscope. This technique made it possible, to anesthetists operating around the world, to define a standard procedure of tracheal intubation in order to place a tube in a trachea, by which a patient can receive oxygen and at the same time can have the air ways protected during the anesthesia.

Despite the improvement of such technique, obtained at a global level by years and years of clinical practice, and despite the different shapes of laryngoscopic blades that have been designed and are available on the market, in some cases direct laryngoscopy is not possible, because of particular anatomical parts in some persons, or because of particular situations of emergency that can occur. For example, it is not possible when the alignment of the three patient's axis (oral axis, pharyngeal axis, and laryngeal axis) cannot be achieved by the standard actions that a physician knows and can put in practice.

For a very long time, there has been a widely recognized need to find some new alternative instruments and techniques, so that an optimal visualization of glottis can be achieved, especially in cases that direct laryngoscopy is not effective or even impossible.

When new technologies became available, with new advanced materials, the laryngeal structures had more chances to be properly visualized, even in an indirect way, by the so called indirect laryngoscopy.

Therefore, the techniques of indirect laryngoscopy made it possible to visualize the laryngeal structures clearly, using a camera or an optical fiber, or even a system of prisms with the distal elements placed close to the glottis.

Nowadays, a large set of instruments are available, permitting one to handle difficult situations, and many of these instruments embed some systems based on indirect laryngoscopy.

The important event that caused a big acceleration in progress of this technique, in the field of anesthesiology when diffusion was still at the beginning, surely has been the construction and marketing of optical fibers. Since the optical fibers started to become available in the clinical practice, the flexible fiberscope has become the reference instrument applied to all difficult intubation cases, even considering that some new professional operators had to be trained, said operators coming from the field of experts in anesthesiology, the otolaryngologists, and the surgeons specialized in chest.

The attempts to design less complex devices, suitable to be used by common anesthetists in the clinical practice, and suitable to provide at the same time the same advantages of optical fibers in indirect laryngoscopy, have led to design of some new special instruments. These are based on standard laryngoscopes, that have a blade which is a part that can move the tongue and to lift/carry the epiglottis, having a particularly curved profile, where an optical fiber is embedded, said fiber reaching the far end of the same blade.

The Bullard laryngoscope was the first of this kind of laryngoscopes, providing the possibility of inspection of laryngeal structures, once the obstacle of the anatomical curve, preventing the direct vision of glottis, has been easily and safely overcome, with a simultaneous precise support of soft tissues, so that an orotracheal intubation can be suddenly achieved.

With reference to devices that, at the present time, permit the achievement of the indirect laryngoscopy technique, except the flexible and stiff fiberscope, or Bonfils fiberscope, two possible classes can be recognized:
the videolaryngoscopes (Glidescope®, McGrath®, and C-MAC®); [00013] the tunneled-systems (Upscher Scope®, Bullard laryngoscope, Airtraq®, Pentax AWS®, and A.P. Advance LMA®). Besides the different visual, optical or video systems, each device belongs to the first or the second class, according to the procedure of using the same device, when the operator can see the laryngeal structures and should insert the endotracheal tube until its proper position in trachea.

Devices in the first class, Glidescope®, McGrath®, and C-MAC®, once the indirect laryngoscopy is possible, according to the blade's shape, usually require that the operator, before inserting the endotracheal tube, has previously and properly fixed its shape. Therefore, the tube's profile is adapted, according to a specific angle, by inserting a stiff stylet inside, so that its final shape allows for addressing the tube beyond the tongue, and towards the patient's trachea.

Instead, devices in the second class, like i.e. Airtraq®, Pentax AWS®, and A.P. Advance LMA®, contain the endotracheal tube in a tunnel that is integrated in the blade, so that a guidance can be achieved all along the path, until the tube exits directly with the far end at the proper position in the trachea. Therefore, the procedure does not require the operator to use a stylet, and does not require maneuvering the endotracheal tube either, it just requires the tube to be pushed inside a stiff driving channel.

Although this solution prevents possible risks arising from the use of stylets, and therefore represents a more comfortable procedure of orotracheal intubation, according to an indirect laryngoscopy technique, it is, however, characterized by significant drawbacks.

First of all, the operator is forced to express some strong actions of pulling and rotating the device, and that is just an attempt, sometimes not effective, to align properly the far end of the instrument to the tracheal axis.

Furthermore, the requirement to insert the endotracheal tube in an operative channel, in order to follow a proper driving path, leads as a consequence to the definition of an increased instrument's thickness, and to an increased minimum inter-foreteeth distance, so that the same instrument can enter into the patient's mouth, and/or leads to the use of different instruments of different size, according to the diameter of the tube to be inserted.

### BRIEF SUMMARY OF THE INVENTION

Therefore, the subject of the present invention consists of a device overcoming all the previous drawbacks, and achieving a great versatility of use, because it can be embedded into a large part of the available systems supporting the orotracheal intubation actually on the market, like i.e.: laryngoscopes, video-laryngoscopes, tunneled video-laryngoscopes, etc.

In particular, the device of this invention achieves a guiding and routing system for an endotracheal tube by use of magnetic means, without requiring a direct operative channel especially designed for that, and without requiring a use of a stylet, preventing therefore possible risks of damages for the patient, arising from the same instrument.

Furthermore, the far end of this device includes an articulated joint mechanism, that is useful in case of need to move tissues and anatomical inner parts of patient, or just in case it is necessary to change the curvature of the blade at the distal part, in order to adapt the instrument's shape to the clinical circumstances; or further, in case it is necessary to change the curvature of the blade and at the same time it is necessary that the endotracheal tube follows this new curvature, at the moment when it is pushed down along the profile of the blade, so that the best alignment of distal end of the endotracheal tube and tracheal axis can be achieved.

The most significant characteristic of the present invention is to include a fine tuning of the position of magnetic means, so that it can be fix the “escape angle” of the endotracheal tube from the guiding and routing system, and the intubation procedure can be adapted to different operative circumstances, especially in cases where a direct or indirect visual perception of the situation is possible. Another significant characteristic of the invention is that the same magnetic elements (double function of the magnetic platform) can be used not only as a guiding and routing system for the endotracheal tube, but as a set of elements mechanically connected to the distal and articulated segment of blade, so that is possible to increase the curvature of both blade and endotracheal tube, if necessary.

The disclosed device permits therefore achievement of a significant decrease of thickness, that define the minimal inter-foreteeth distance in order to use the actual tunneled laryngoscopes, so that they can be inserted in a patient's mouth, a variable routing direction at each step of progress, under control by a tuning mechanism, a more comfortable procedure that does not require any use of a stylet to define shape and profile, and the possibility to move the distal end by an articulated joint mechanism, that is under control using the same guiding and routing system for the endotracheal tube.

Therefore, it is specific subject of the present invention a device comprising a set of magnetic elements, particularly suitable as a support to the procedure of a patient's tracheal intubation, and preferably embedded in a blade of a laryngoscope, or an Airtraq® video-laryngoscope, or a Glidescope® video-laryngoscope, or a LMA® video-laryngoscope, or other similar instruments, and characterized in that comprising:
a first set of magnetic elements that are all connected to each other, by a first flexible/articulated supporting structure, in order to achieve a so called magnetic platform, free to move forward or backward, under control of an operator, along the longitudinal direction inside an operative channel belonging to the same device;
a second set of magnetic elements that are all connected to each other, by a second flexible supporting structure, in order to achieve a so called magnetic train, previously inserted in an endotracheal tube; elements of magnetic train have an opposite polarity in respect of elements of the magnetic platform, so that a magnetic attractive force is generated by their interaction for all the time, keeping said endotracheal tube strictly in contact with said magnetic platform, so that the same endotracheal tube can move, forward or backward, under control of an operator, along the longitudinal profile of said operative channel, in order to achieve a guiding and routing system for the endotracheal tube, because the tube follows a curved trajectory and it extends itself with a far end aligned to a direction that follows the tangent to the magnetic platform profile, at the last point of magnetic contact.

### BRIEF DESCRIPTION OF THE SEVERAL VIEWS OF THE DRAWINGS

The present invention will now be described for illustrative but not !imitative purposes, according to its preferred embodiments, with particular reference to figures of the enclosed drawings, wherein:

FIG. 1 is a front perspective view of a device with magnetic elements, according to the present invention, preferably embedded in a Airtraq® video-laryngoscope;

FIG. 2 is a lateral view of a device with magnetic elements embedded in a Airtraq® video-laryngoscope, like that of FIG. 1, inserted in the upper part of a patient, related to mouth and first tract of air ways, the same patient being represented according to a lateral sectioned view;

FIGS. 3, 4 and 5 are a sequence of front perspective views of the same device of FIG. 1, and of an endotracheal tube that is moved in contact with the so called magnetic platform part, and that is represented at three different positions;

FIGS. 6, 7 and 8 are a sequence of lateral perspective views of the same device of FIG. 1, where the so called magnetic platform is moved forward, and it is represented at three different positions;

FIG. 9 is a front perspective view of a device with magnetic elements, according to the present invention, preferably embedded in a standard laryngoscope;

FIG. 10 is a lateral view of a device with magnetic elements embedded in a standard laryngoscope, like that of FIG. 9, inserted in the upper part of a patient, related to mouth and first tract of air ways, the same patient being represented according to a lateral sectioned view;

FIG. 11 is a front perspective view of a device with magnetic elements, according to the present invention, preferably embedded in a LMA® video-laryngoscope;

FIG. 12 is a front perspective view of a device with magnetic elements, according to the present invention, preferably embedded in a LMA® video-laryngoscope, where position of magnetic platform can be moved under control of an operator;

FIG. 13 is a lateral view of a device with magnetic elements embedded in a LMA® video-laryngoscope, like that of FIG. 11, inserted in the upper part of a patient, related to mouth and first tract of air ways, the same patient being represented according to a lateral sectioned view;

FIG. 14 is a lateral view of a device with magnetic elements embedded in a Airtraq® video-laryngoscope, like that of FIG. 1, where the forward position of magnetic platform defines an outgoing angle α of the endotracheal tube;

FIG. 15 is a lateral view of a device with magnetic elements embedded in a Airtraq® video-laryngoscope, like that of FIG. 1, where the backward position of magnetic platform defines an outgoing angle β of the endotracheal tube;

FIG. 16 is a front view of a device with magnetic elements embedded in a Airtraq® video-laryngoscope, like that of FIGS. 14 and 15, where it is shown the presence of an optical visual channel;

FIG. 17 is a front view of a detail of FIG. 16, with reference to the distal end of the outgoing part of an endotracheal tube, with respective optical lighting and inspection means of a Airtraq® video-laryngoscope.

### DETAILED DESCRIPTION

It is here underlined that only few of the many conceivable embodiments of the present invention are described, which are just some specific non-limiting examples, having the possibility to describe many other embodiments based on the disclosed technical solutions of the present invention.

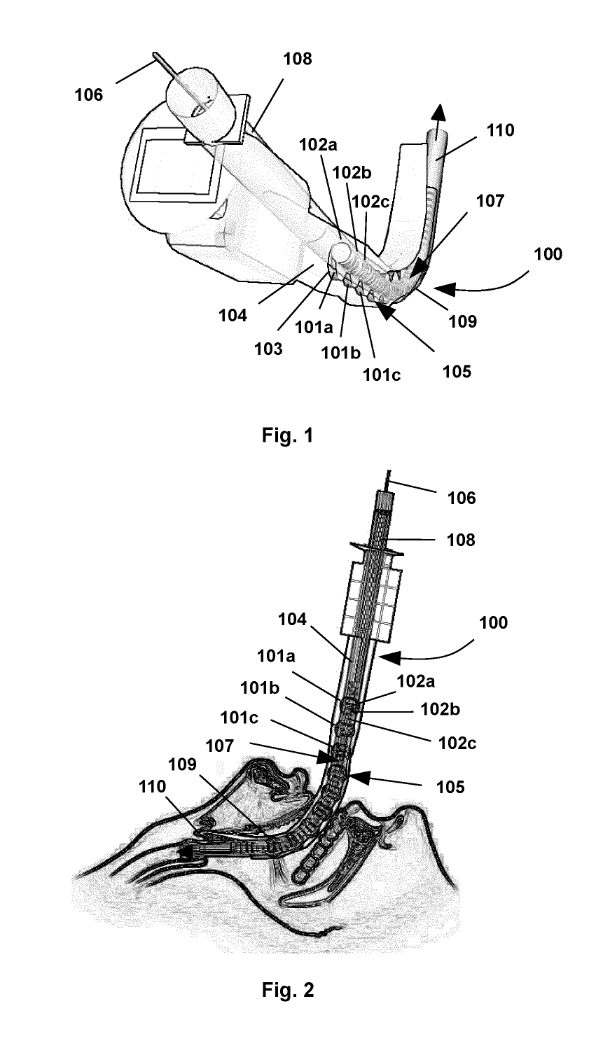

In FIG. 1 it is illustrated a device with magnetic elements 100, particularly suitable as a support to the procedure of tracheal intubation of a patient, embedded in the blade of a Airtraq® video-laryngoscope. The blade of the same laryngoscope, in the prior art has a longitudinal profile with some protruding walls, that achieves a guiding and routing tunnel, along which an endotracheal tube 108 is inserted until its final position, with the distal end in trachea, as illustrated in FIG. 2.

Instead, device 100 of the present invention is characterized by having the protruding walls removed, and the guiding function is achieved through a magnetic platform 105, that is placed into the blade, and that interacts with a magnetic train 107, that has been previously and steadily inserted within an endotracheal tube 108.

The magnetic platform 105 comprises a first set of magnetic elements 101a, 101b, . . . , etc., that are all connected each other, by a first flexible/articulated supporting structure 103; the magnetic train 107 comprises instead a second set of magnetic elements 102a, 102b, . . . , etc., that are all connected each other, by a second flexible supporting structure 106. The elements 102a, 102b, . . . , etc., of the magnetic train 107 have an opposite polarity in respect of elements 101a, 101b, . . . , etc., of said magnetic platform 105, so that a magnetic attractive force is generated by interaction all the time, keeping said endotracheal tube 108 strictly in contact with said magnetic platform 105.

The magnetic platform 105 is free to move forward or backward, under control of an operator, along the longitudinal direction of an operative channel 104 belonging to the same device 100. Furthermore, the magnetic platform 105 can be completely removed from its position inside the operative channel 104, when said structure 103 (after disconnection of gear wheel 120) is strongly pulled out by the operator, so that the entire device 100 can be removed in a very safe way and without trauma for patient, once the endotracheal tube 108 has been properly inserted in trachea.

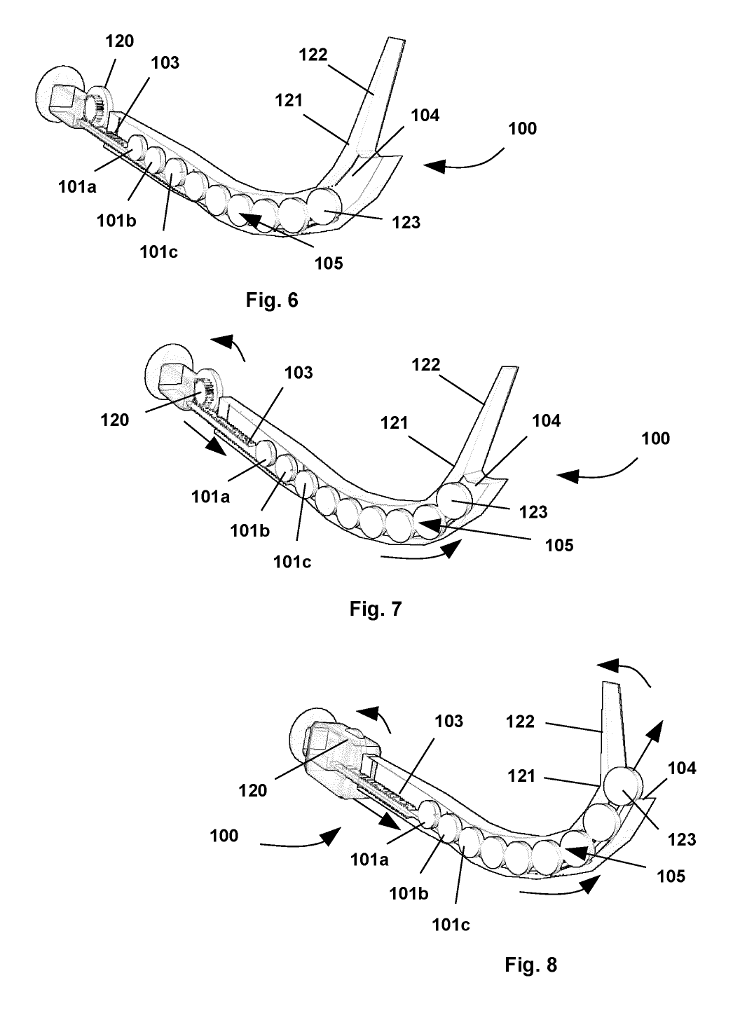

FIGS. 6, 7 and 8 show a sequence where the so called magnetic platform 105 is moved, and it is represented at three different positions.

The supporting structure 103 of the magnetic platform 105 is composed of a set of elements, that connect all respective magnetic elements 101a, 101b, . . . , etc., and said set of elements are stiff in the longitudinal direction and articulated laterally. The same structure 103 ends, at the closest part to the human operator, with a gear wheel 120; when the operator makes the wheel 120 rotate backward, the magnetic platform 105 goes ahead deep in the blade, when the operator makes the wheel 120 to rotate forward, the magnetic platform 105 goes back to the closest part to operator. The gear wheel 120 includes a special stopping/blocking system that makes it impossible, if activated, for the magnetic platform 105 to move along the operative channel 104.

The supporting structure 106 of the magnetic train 107 is composed of a set of elements, that connect all respective magnetic elements 102a, 102b, . . . , etc., and said set of elements are stiff in the longitudinal direction and articulated laterally. When the operator pushes the structure 106 forward, the magnetic train 107 goes ahead into the endotracheal tube 108, when the operator pushes the structure 106 backward, the magnetic train 107 goes back from the endotracheal tube 108, to the closest part to operator.

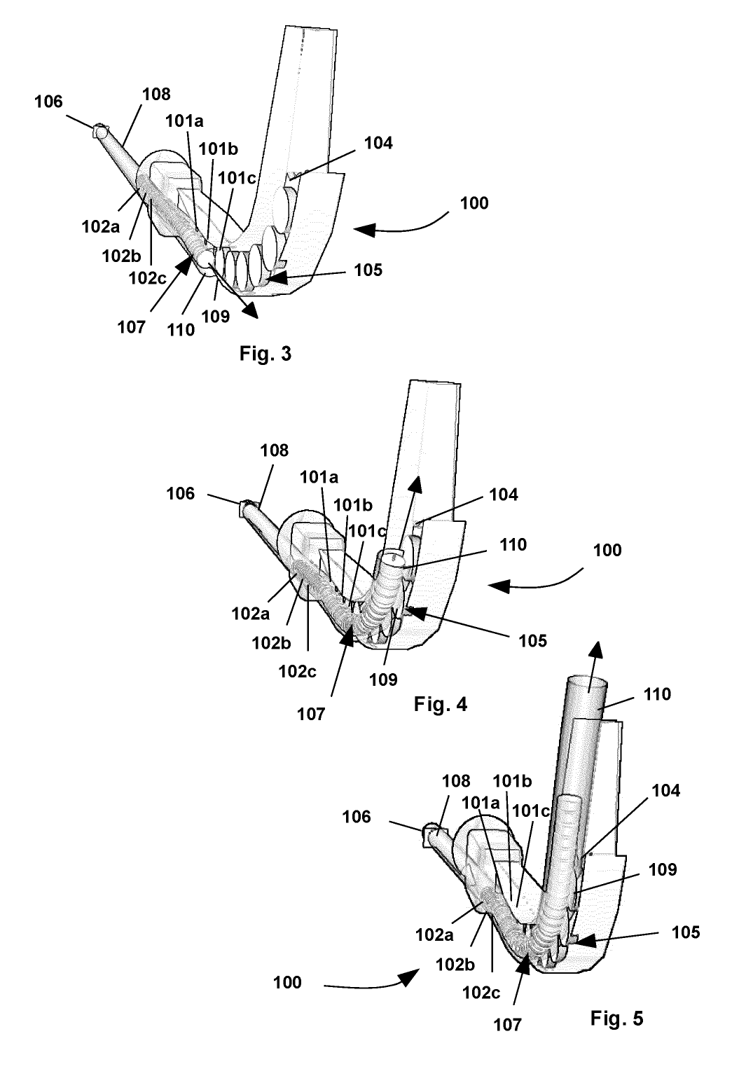

FIGS. 3, 4 and 5 show a sequence where an endotracheal tube 108 is moved in contact with the magnetic platform 105, and it is represented at three different positions.

An operator can move the endotracheal tube 108, forward or backward, along the longitudinal profile of said operative channel 104, in order to achieve a guiding and routing system of the same endotracheal tube 108, because the tube follows a respective curved trajectory and it extends itself with its far end 110 aligned to a direction that follows the tangent to said operative channel's 104 profile, at the last point of magnetic contact 109.

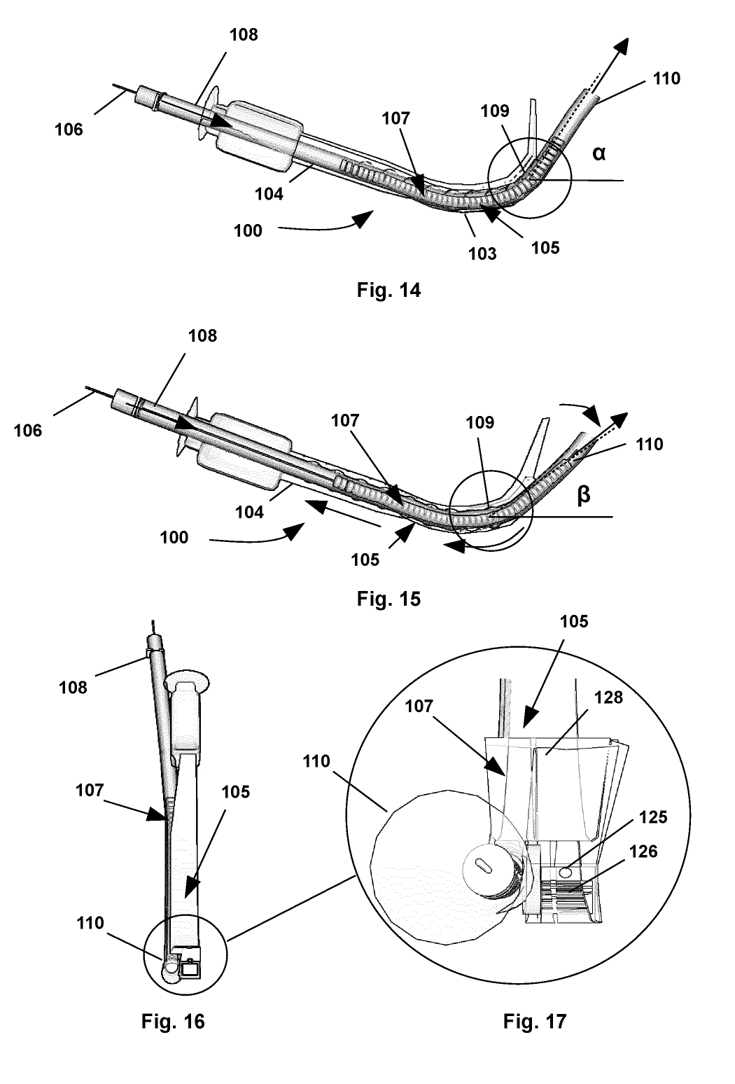

FIG. 14 shows that a forward position of magnetic platform 105, with a forward last point of magnetic contact 109, defines an outgoing angle α of the endotracheal tube that is very diverging in respect to an horizontal line; instead, FIG. 15 shows that a backward position of magnetic platform 105, with a backward last point of magnetic contact 109, defines an outgoing angle β<α of the endotracheal tube 108 that is less diverging in respect to the same horizontal line.

The elements 101a, 101b, . . . , etc., of the magnetic platform 105 have a cylindrical shape and are placed transversally in respect to the longitudinal direction of said operative channel 104 where they are inserted, so that they can roll inside, and so that they achieve a little friction of magnetic platform 105 during its motion.

The elements 102a, 102b, . . . , etc., of the magnetic train 107 have a cylindrical shape and are placed coaxially in respect to the longitudinal direction of said endotracheal tube 108 where they are inserted, so that they can change direction in order to follow a curved shape of the endotracheal tube 108, when it follows a specific path in contact with the magnetic platform 105.

The first set of magnetic elements 101a, 101b, . . . , etc., can be entirely composed of magnetic elements, or otherwise can be composed of an alternation of magnetic elements and magnetically neutral elements, i.e. made of plastic, so that the latter ones have as unique function to keep at a specific distance the elements of said magnetic platform 105.

The second set of magnetic elements 102a, 102b, . . . , etc., can be entirely composed of magnetic elements, or otherwise can be composed of an alternation of magnetic elements and magnetically neutral elements, i.e. made of plastic, so that the latter ones have as unique function to keep at a specific distance the elements of said magnetic train 107.

The elements 101a, 101b, . . . , etc., of the magnetic platform 105, and the elements 102a, 102b, . . . , etc., of the magnetic train 107, can be composed of permanent magnets or electromagnets with a related power supply circuit, and batteries with on/off switches and tuning devices.

According to another embodiment of the invention, the far end 122 of the blade of device 100 is articulated, and can rotate clockwise or counterclockwise in respect to a transversal pivot 121. The last element 123 of the magnetic platform 105 is composed of a magnetically neutral element, i.e. made of plastic, so that a motion forward of the magnetic platform 105 makes the last element 123 push against the element 122, and so that the same element 122 is lifted up and allows the displacement of patient's tissues and/or other anatomical parts—this is called McCoy function. The same element 122 comprises a return spring, that permits the element 122 to go back to its initial position, when the magnetic platform 105 is moved backward and the element 123 stops to make pressure on the side, and goes back to its initial position also. An elastic membrane of protection, placed at the terminal part of the operative channel 104, achieves two results: first, it permits at least two elements belonging to the magnetic platform 105 to go outside from the distal part of the operative channel 104; and second, it keeps the magnetic platform 105 sterilized, so that it can be used on another blade that is disposable, or on similar means using this type of technology.

The device 100 of the present invention can comprise, as accessories, a first disposable covering, preferably made of plastics and sterilized, that covers the blade of device 100 and all the magnetic platform 105, and a second disposable covering, also preferably made of plastics and sterilized, that covers all the magnetic train 107, placed inside the endotracheal tube 108, so that to prevent possible infections and/or contaminations. Indeed, the device 100 itself can be disposable, with reference to the entire blade and channels 104 and 128, including the magnetic platform 105 and further video or optical devices eventually embedded.

The device 100 of the invention can be easily integrated with the optical visual means, typical of an Airtraq® video-laryngoscope.

In example, FIGS. 16 and 17 show said operative channel 104, where said magnetic platform 105 can move inside, exactly parallel to an optical visual channel 128, through which the visual signals of the detected image are transmitted, from the far end of device 100 to the view of said human operator. Furthermore, it is possible that visual channel 128 is just an empty channel, where visual systems like a fiberscope, or other available devices, can be inserted.

Furthermore, some embodiments of the invention disclose that said operative channel 104 is placed at the right side, from the point of view of an operator, in respect to said optical visual channel 128, or at the left side in order to make it comfortable to left-handed operators. Then, another embodiment of invention discloses that said operative channel 104 and said optical visual channel 128 are placed side by side, with the endotracheal tube 108 placed in the space between.

In such a way, the patient's anatomical parts result in being better protected, in respect to situations where the endotracheal tube 108 could touch the tongue, and the tissues can be entrapped between the same endotracheal tube 108 and the elements of device 100, leading as a consequence to risks of abrasions and inner scratching to the patient.

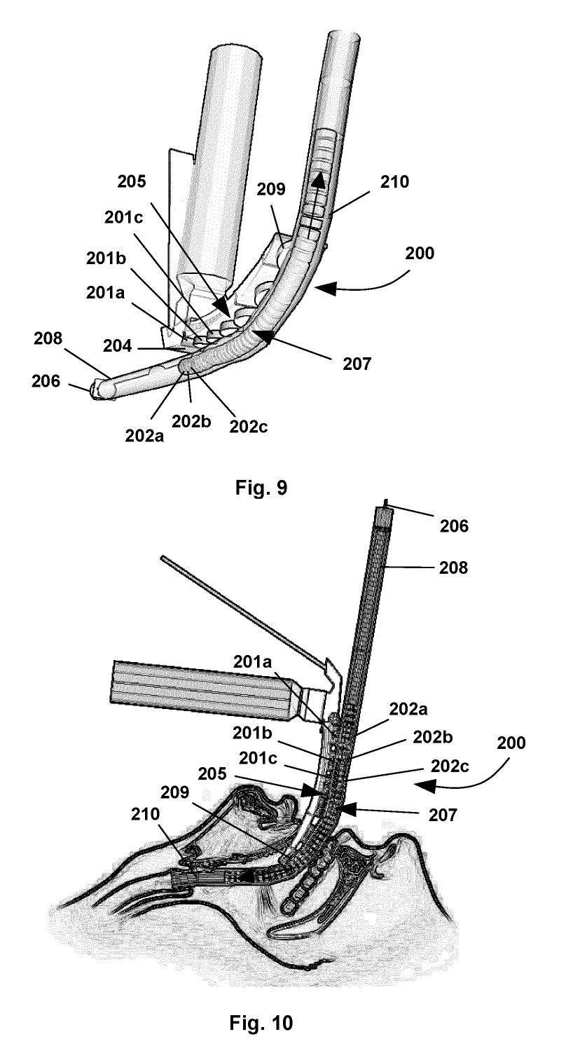

FIG. 9 shows a device with magnetic elements 200, preferably embedded in a standard laryngoscope. The guiding function is achieved through a magnetic platform 205, that is placed at the bottom of the blade, and that interacts with a magnetic train 207, that has been previously and steadily inserted within an endotracheal tube 208.

The magnetic platform 205 comprises a first set of magnetic elements 201a, 201b, . . . , etc., that are all connected to each other, directly to the blade of laryngoscope; the magnetic train 207 comprises instead a second set of magnetic elements 202a, 202b, . . . , etc., that are all connected to each other, by a flexible supporting structure 206. The elements 202a, 202b, . . . , etc., of the magnetic train 207 have an opposite polarity in respect of elements 201a, 201b, . . . , etc., of said magnetic platform 205, so that a magnetic attractive force is generated by interaction all the time, keeping said endotracheal tube 208 strictly in contact with said magnetic platform 205.

An operator can move the endotracheal tube 208, forward or backward, along the longitudinal profile of the blade of laryngoscope, in order to achieve a guiding and routing system of the same endotracheal tube 208, because the tube follows a respective curved trajectory and it extends itself with its far end 210 aligned to a direction that follows the tangent to the blade's profile, at the last point of magnetic contact 209.

Therefore, the device with magnetic elements 200 achieves a guiding and driving path, along which an endotracheal tube 208 is pushed up to its final position, with the distal end in trachea, as illustrated in FIG. 10.

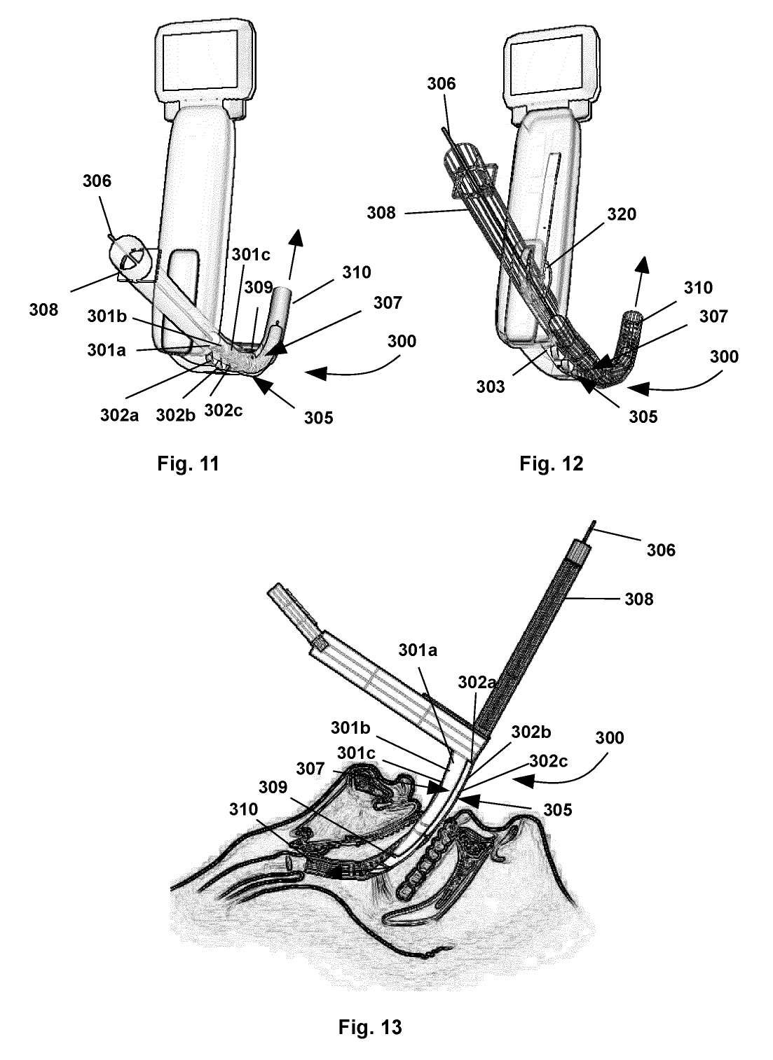

FIG. 11 shows a device with magnetic elements 300, preferably embedded in a LMA® video-laryngoscope. The guiding function is achieved through a magnetic platform 305, that is placed at a lateral side of the blade, and that interacts with a magnetic train 307, that has been previously and steadily inserted within an endotracheal tube 308.

The magnetic platform 305 comprises a first set of magnetic elements 301a, 301b, . . . , etc., that are all connected each other, and that are placed: inside the same structure housing the video or optical devices of laryngoscope and supporting the disposable and interchangeable blade; or inside the same disposable blades that are assembled on said structure according to different clinical circumstances; the magnetic train 307 comprises instead a second set of magnetic elements 302a, 302b, . . . , etc., that are all connected to each other, by a flexible supporting structure 306. The elements 302a, 302b, . . . , etc., of the magnetic train 307 have an opposite polarity in respect of elements 301a, 301b, . . . , etc., of said magnetic platform 305, so that a magnetic attractive force is generated by interaction all the time, keeping said endotracheal tube 308 strictly in contact with said magnetic platform 305.

As shown in FIG. 12, the magnetic platform 305 is free to move forward or backward, under control of an operator, along the longitudinal direction of an operative channel 304 belonging to the same device 300.

The supporting structure 303 of the magnetic platform 305 is composed of a set of elements, that connect all respective magnetic elements 301a, 301b, . . . , etc., and said set of elements are stiff in the longitudinal direction and articulated laterally. The same structure 303 ends, at the closest part to the human operator, with a gear wheel 320; when the operator makes the wheel 120 rotate backward, the magnetic platform 305 goes ahead deep in the blade, when the operator makes the wheel 320 rotate forward, the magnetic platform 305 goes back to the closest part to operator.

The operator can move the endotracheal tube 308, forward or backward, along the longitudinal profile of the blade of laryngoscope, in order to achieve a guiding and routing system of the same endotracheal tube 308, because the tube follows a respective curved trajectory and it extends itself with its far end 310 aligned to a direction that follows the tangent to the blade's profile, at the last point of magnetic contact 309.

Therefore, the device with magnetic elements 300 achieves a guiding and routing path, along which an endotracheal tube 308 is pushed up to its final position, with the distal end in trachea, as illustrated in FIG. 13.

Also in this case, the device 300 of the present invention can comprise, as accessories, a first disposable covering, preferably made of plastics and sterilized, that covers the blade (or the structure supporting the blade) of device 300 and all the magnetic platform 305, and a second disposable covering, also preferably made of plastics and sterilized, that covers all the magnetic train 307, placed inside the endotracheal tube 308, so that to prevent possible infections and/or contaminations.

Therefore, the above examples show that the present invention achieves all the proposed objectives. In particular, it discloses a device that permits one to overcome all the drawbacks of the prior art, achieving a great versatility of use, because it can be embedded into a large part of the available systems supporting the orotracheal intubation actually on the market, like i.e.: laryngoscopes, video-laryngoscopes, tunneled video-laryngoscopes, etc.

In particular, the device of this invention achieves a guiding and routing system for an endotracheal tube by use of magnetic means, without requiring a direct operative channel especially designed for that, and without requiring a use of a stylet, preventing therefore possible risks of damages for the patient, arising from the same instrument.

Furthermore, the far end of this device includes an articulated joint mechanism, that is useful in case of need to move tissues and anatomical inner parts of patient, or just in case it is necessary to change the curvature of the blade at the distal part, in order to adapt the instruments shape to the clinical circumstances; or further, in case it is necessary to change the curvature of the blade and at the same time it is necessary that the endotracheal tube follows this new curvature, at the moment when it is pushed down along the profile of the blade, so that the best alignment of the distal end of the endotracheal tube and tracheal axis can be achieved.

The most significant characteristic of the present invention is to include a fine tuning of the position of magnetic means, so that it can fix the “escape angle” of the endotracheal tube from the guiding and routing system, and the intubation procedure can be adapted to the different operative circumstances, especially in cases where a direct or indirect visual perception of the situation is possible. Another significant characteristic of the invention is that the same magnetic elements (double function of the magnetic platform) can be used not only as a guiding and routing system for the endotracheal tube, but as a set of elements mechanically connected to the distal and articulated segment of blade, so that is possible to increase the curvature of both blade and endotracheal tube, if necessary.

The disclosed device permits therefore achievement of a significant decrease of thickness, that define the minimal inter-foreteeth distance in order to use the actual tunneled laryngoscopes, so that they can be inserted in a patient's mouth, a variable routing direction at each step of progress, under control by a tuning mechanism, a more comfortable procedure that does not require any use of a stylet to define shape and profile, and the possibility to move the distal end by an articulated joint mechanism, that is under control using the same guiding and routing system for the endotracheal tube.

The present invention has been described for illustrative but not limitative purposes, according to its preferred embodiments, but it is clear that modifications and/or changes can be introduced by those skilled in the art without departing from the relevant scope, as defined in the enclosed claims.

1. A device having a set of magnetic elements, particularly suitable as a support to procedure of patient's tracheal intubation, and preferably embedded in a blade of a laryngoscope, or an Airtraq® video-laryngoscope, or a Glidescope® video-laryngoscope, or a LMA® video-laryngoscope, or other similar instruments, and comprising:
a first set of magnetic elements, that are all connected to each other, by a first flexible/articulated supporting structure, in order to achieve a so called magnetic platform, free to move forward or backward, under control of an operator, along the longitudinal direction of an operative channel belonging to the same device;
a second set of magnetic elements, that are all connected to each other, by a second flexible supporting structure, in order to achieve a so called magnetic train, previously and steadily inserted in an endotracheal tube; with the elements of said magnetic train having an opposite polarity in respect of elements of said magnetic platform, so that a magnetic attractive force is generated by interaction all the time, keeping said endotracheal tube strictly in contact with said magnetic platform,
so that the same endotracheal tube can move, forward or backward, under control of an operator, along the longitudinal profile of said operative channel, in order to achieve a guiding and routing system of the endotracheal tube, because the tube follows a respective curved trajectory and it extends itself with its far end aligned to a direction that follows the tangent to said magnetic platform profile, at a last point of magnetic contact.

2. A device having a set of magnetic elements and being particularly suitable as a support to procedure of patient's tracheal intubation, according to claim 1, and being further characterized in that:
said first set of magnetic elements is entirely composed of magnetic elements or otherwise is composed of an alternation of magnetic elements and magnetically neutral elements so that the latter ones have as a unique function to keep at a specific distance the elements of said magnetic platform.

3. A device having a set of magnetic elements particularly suitable as a support to procedure of patient's tracheal intubation, according to claim 1, and being further characterized in that:
said second set of magnetic elements is entirely composed of magnetic element or otherwise is composed of an alternation of magnetic elements and magnetically neutral elements so that the latter ones have as a unique function to keep at a specific distance the elements of said magnetic train.

4. A device having a set of magnetic elements and being particularly suitable as a support to procedure of patient's tracheal intubation, according to claim 1, and being further characterized in that:
said elements of the magnetic platform have a cylindrical shape and are placed transversally in respect to the longitudinal direction of said operative channel where they are inserted, so that they can roll inside, and so that they achieve a little friction of magnetic platform during its motion.

5. A device having a set of magnetic elements and being particularly suitable as a support to procedure of patient's tracheal intubation, according to claim 1, and being further characterized in that:
said elements of the magnetic train have a cylindrical shape and are placed coaxially in respect to the longitudinal direction of said endotracheal tube where they are inserted, so that they can change direction in order to follow a curved shape of the endotracheal tube, when it follows a specific path in contact with the magnetic platform.

6. A device having a set of magnetic elements and being particularly suitable as a support to procedure of patient's tracheal intubation, according to claim 1, and being further characterized in that:
said supporting structure of the magnetic platform is composed of a set of elements, that connect all respective magnetic elements and said set of elements are stiff in the longitudinal direction and articulated laterally; the same structure ends, at the closest part to the human operator, with a gear wheel, such that when the operator makes the wheel to rotate backward, the magnetic platform goes ahead deep in the blade, and when the operator makes the wheel rotate forward, the magnetic platform goes back to the closest part to operator.

7. A device having a set of magnetic elements and being particularly suitable as a support to procedure of patient's tracheal intubation, according to claim 1, and being further characterized in that:
said supporting structure of the magnetic train is composed of a set of elements, that connect all respective magnetic elements and said set of elements are stiff in the longitudinal direction and articulated laterally such that when the operator pushes the structure forward, the magnetic train goes ahead into the endotracheal tube, and when the operator pushes the structure backward, the magnetic train goes back from the endotracheal tube to the closest part to operator.

8. A device having a set of magnetic elements and being particularly suitable as a support to procedure of patient's tracheal intubation, according to claim 1, and being further characterized in that:
the far end of the blade of device is articulated, and can rotate clockwise or counterclockwise in respect to a transversal pivot, the last element of the magnetic platform is composed of a magnetically neutral element, so that a motion forward of the magnetic platform makes the last element to push against the side of the element, and so that the same element is lifted up and allows the displacement of patient's tissues and/or other anatomical parts—this is called McCoy function;
and the same element comprises a return spring, that permits the element to go back to its initial position, when the magnetic platform is moved backward and the element stops to make a pressure on the element, and goes back to its initial position also;
and whereby an elastic membrane of protection, placed at the terminal part of the operative channel, achieves two results: first, it permits at least two elements belonging to the magnetic platform to go outside from the distal part of the operative channel; and second, it keeps the magnetic platform sterilized, so that it can be used on another blade that is disposable, or on similar means using this type of technology.

9. A device having a set of magnetic elements and being, particularly suitable as a support to procedure of patient's tracheal intubation, according to claim 1, and being further characterized in that:
said elements of the magnetic platform are composed of permanent magnets or electromagnets with a related power supply circuit, and batteries with on/off switches and tuning devices;
said elements of the magnetic train are composed of permanent magnets or electromagnets with a related power supply circuit, and batteries with on/off switches and tuning devices.

10. A device having a set of magnetic elements and being particularly suitable as a support to procedure of patient's tracheal intubation, according to claim 1, and further comprising:
a first disposable covering, preferably made of plastics and sterilized, that covers the blade of the device and all of the magnetic platform, so as to prevent possible infections and/or contaminations;
the device itself being disposable, with reference to the entire blade and channels, including the magnetic platform and further video or optical devices eventually embedded; and
a second disposable covering, preferably made of plastics and sterilized, that covers all the magnetic train once placed inside the endotracheal tube, so as to prevent possible infections and/or contaminations.

11. A device having a set of magnetic elements and being particularly suitable as a support to procedure of patient's tracheal intubation, according to claim 1, and being further characterized in that:
said operative channel, wherein said magnetic platform can move inside, is positioned exactly parallel to an optical visual channel, through which the visual signals of the detected image are transmitted, from the far end of device to the view of said human operator.

12. A device having a set of magnetic elements and being particularly suitable as a support to procedure of patient's tracheal intubation, according to claim 1, and further characterized in that:
said operative channel is placed at one of the right side, from the point of view of an operator, in respect to said optical visual channel or the left side, in order to make it comfortable to left-handed operators.

13. A device having a set of magnetic elements and being particularly suitable as a support to procedure of patient's tracheal intubation, according to claim 1, and being further characterized in that:
said operative channel and an optical visual channel are placed side by side, with the endotracheal tube placed in the space between,
so that the patient's anatomical parts result in being better protected, in respect to situations where the endotracheal tube could touch the tongue or the tissues could be entrapped between the same endotracheal tube and the elements of device.

14. A device having a set of magnetic elements and being particularly suitable as a support to procedure of patient's tracheal intubation, according to claim 6, and being further characterized in that:
said magnetic platform can be completely removed from its position inside the operative channel when said structure is strongly pulled out by operator, after disconnection of gear wheel,
so that the entire device can be removed in a very safe way and Without trauma for the patient, once the endotracheal tube has been properly inserted in trachea.

15. A device having a set of magnetic elements and being particularly suitable as a support to procedure of patient's tracheal intubation, according to claim 6, and being further characterized in that:
said gear wheel includes a special stopping/blocking system that makes it impossible, if activated, for the magnetic platform to move along the operative channel.

16. A device for use in tracheal intubation in conjunction with a blade of a laryngoscope or other similar instrument, said device comprising:
a magnetic platform; said platform having a first set of magnetic elements connected together by a first flexible or articulated supporting structure, whereby the platform is free to move forward or backward, under control of an operator, along a longitudinal direction of an operative channel belonging to the same device;
a magnetic train; said train having a second set of magnetic elements connected together by a second flexible or articulated supporting structure; said second set of magnetic elements having an opposite polarity with respect to the first set of magnetic elements, whereby when the train is inserted in an endotracheal tube and the platform is positioned against the endotracheal tube, a magnetic attractive force is generated between the train and the platform, whereby the endotracheal tube is kept in contact with the platform;
such that the endotracheal tube can move, forward or backward, under control of an operator, along the longitudinal direction of said operative channel, in order to achieve a guiding and routing system of the endotracheal tube.
